(19) United States
(12) Patent Application Publication
(10) Pub. No.: US 2005/0182030 A1
(43) Pub. Date: Aug. 18, 2005

(54) Liquid injectable formulation of disodium pamidronate

(75) Inventors: Dennis Szymanski, Winfield, IL (US); Sam Hahn, Scarborough (CA)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: American Pharmaceutical Partners, Inc., Schaumburg, IL (US)

(21) Appl. No.: 11/036,295

(22) Filed: Jan. 14, 2005

Related U.S. Application Data
(63) Continuation of application No. 10/346,924, filed on Jan. 17, 2003, now abandoned.
Application No. 10/346,924 is a continuation of application No. 10/138,170, filed on May 3, 2002, now abandoned.

(30) Foreign Application Priority Data
May 10, 2001 (CA) 2,347,330

(51) Int. Cl. A61K 31/66

(52) U.S. Cl. 514/102

(57) ABSTRACT

The present invention relates to an improved injectable ready-to-use preparation of pamidronate salts, methods for its manufacture and uses of the solution of the invention in the manufacture of pharmaceutical compositions for the treatment of diseases selected from the group of tumour-induced hypercalcaemia, Paget's disease, osteoporosis, bone metastases, or breast cancer. The ready-to-use solution comprises a physiologically acceptable alkaline salt of pamidronate which is water soluble and a physiologically acceptable aqueous solvent having a concentration of between 0.1 and 100 mg/mL, wherein the solution is provided in a sealed non-reactive plastic container.

### CROSS-REFERENCE TO RELATED PATENT APPLICATIONS

This patent application is a continuation of U.S. patent application Ser. No. 10/346,924, filed on Jan. 17, 2003, which is a continuation of U.S. patent application Ser. No. 10/138,179, filed on May 3, 2002, which claims priority from Canadian Patent Application No. 2,347,330, filed on May 10, 2001.

### FIELD OF THE INVENTION

The present invention relates to an improved injectable ready to use preparation of pamidronate salts of the formula given by

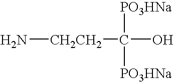

### BACKGROUND OF THE INVENTION

3-amino-1-hydroxypropane-1,1-diphosphonate disodium, the disodium salt of pamidronic acid, is a well-known compound useful as a bone resorption inhibitor. Also known as pamidronate, pamidronate disodium or disodium pamidronate, the compound is part of the therapeutic class of compounds called bisphosphonates. Bisphosphonates used as inhibitors of bone resorption all contain two phosphonate groups attached to a single carbon atom, forming a “P—C—P” structure. The bisphosphonates are therefore stable analogues of naturally occurring pyrophosphate-containing compounds, which now helps to explain their intracellular as well as their extracellular modes of action. The mode of action of bisphosphonates was originally ascribed to physico-chemical effects on hydroxyapatite crystals, a major inorganic component of bone, but it has gradually become clear that cellular effects must also be involved. Bisphosphonates inhibit bone resorption by being selectively taken up and absorbing to mineral surfaces in bone, where they interfere with the action of osteoclasts. It is likely that bisphosphonates are internalized by osteoclasts and interfere with specific biochemical processes and induce apoptosis.

Several bisphosphonates including etidronate, elodronate, pamidronate, alendronate, and risedronate are established as effective treatments in clinical disorders such as Paget's disease of bone, hypercalceamia of a malignancy, and bone metastases. Bisphosphonates are also now well established as successful antiresorptive agents for the prevention and treatment of osteoporosis. Additional indications include the reduction of bone pain associated with certain illnesses and to treat bone loss due to breast cancer. U.S. Pat. Nos. 4,711,880 and 4,639,338 to Stahl et al. disclose the preparation of the crystalline pentahydrate form of disodium pamidronate from pamidronic acid. A heated aqueous suspension of pamidronic acid is partially neutralized with aqueous sodium hydroxide (NaOH) to pH 7 to 7.5. Crystallization is then initiated and the disodium pamidronate is collected by filtration. The pentahydrate comprises about 24.1 to 25% water and the product is stable to storage under approximately normal ambient conditions. The commercially available formulation, AREDIA™, contains the lyophilized form of pamidronate disodium pentahydrate.

Other crystalline forms of disodium pamidronate convert to the pentahydrate depending upon humidity and amount of water present (Stahl et al.) resulting in varying compositions of hydrates. Accordingly, it is difficult to use preformed disodium salts of pamidronic acid (such as anhydrous or partially hydrated forms other than pentahydrate) for further processing into sterile pharmaceuticals due to the interconversion of other crystalline forms of disodium pamidronate.

At present, pamidronate is usually administered intravenously, due to the poor absorption from the gastrointestinal system. Pamidronate is supplied commercially as a lyophilized powder that must be reconstituted with a pharmaceutically acceptable solvent before administration to a patient.

Problems associated with a lyophilized formulation include a risk of microbial contamination during reconstitution and an inability to terminally sterilize the drug product. Double handling of the drug is required, as the lyophilized drug is first required to be reconstituted and then administered. Additionally, time is needed to dissolve the powder and prolonged shaking may be required.

Pamidronate in a liquid formulation has been shown to be unstable/reactive during long-term storage (Canadian patent application 2,141,964). In addition, current guidelines for storage of reconstituted solutions state that the solution should not be kept for more than 24 hours.

One answer to the stability problem is proposed in Canadian patent application 2,141,964, which discloses injection solutions that are stable when stored in glass packaging, where the pH of the injection and solution is about 3.0 to 4.5 and polyethylene glycols are used to stabilize the solution. However, this formulation contains ingredients that are unnecessary for therapeutic purposes, and the process to prepare the formulation requires several steps, such as pH adjustment.

Another liquid formulation of disodium pamidronate is disclosed in U.S. Pat. No. 6,160,165 to Shinal. This formulation is prepared by making a stirred slurry of pamidronic acid in water (pamidronic acid is not soluble in water); adding an aqueous solution of sodium hydroxide to the slurry in an about 2:1 molar ratio of sodium hydroxide to pamidronic acid to yield a solution having visual clarity. The solution is packaged in a sealed container to yield a liquid dosage form of pamidronate. No data is given on its stability. No information is provided on sterilization of the solution to yield a pharmaceutically acceptable product. The patent further discloses a lyophilized form of pamidronate, made by the steps above, filtering the solution and freezing and lyophilizing the filtered solution to yield amorphous, essentially anhydrous disodium pamidronate. This process has the disadvantage of a number of manufacturing steps. Additionally, the liquid composition cannot be stored for long periods of time as reaction of the pamidronate with polyvalent cations will occur when stored in glass vials.

Assuring sterility of the injection solution is always a concern for a manufacturer. Reconstitution introduces the risk of microbial contamination. Although the preferred approach to assurance of sterility of a solution, or the gold standard, is terminal steam sterilization through autoclaving, sterile filtration is used when the compound or formulation is subject to lyophilization or is heat sensitive. (Drugs Directorate Guideline, Chemistry and Manufacturing: New Drugs 1990, Health and Welfare Canada).

Ready-to-use solutions of disodium pamidronate, provided in a sealed container, have not been commonly available. Accordingly, there is a need for a stable, ready to use liquid injectable formulation that can be stored at room temperature and does not require reconstitution from a lyophilizate. There is a need for a solution that can be terminally sterilized. There is also a need for a simplified process for making a stable liquid formulation of disodium pamidronate that does not require pH adjustment nor any expensive freeze drying step.

### BRIEF SUMMARY OF THE INVENTION

It is an object of the present invention to provide a storage stable, ready to use solution containing a pharmaceutically acceptable water-soluble alkaline salt of pamidronate as well as a process for its manufacture. In a preferred embodiment, the pharmaceutically acceptable water soluble alkaline salt is the disodium salt.

According to an aspect of the invention, there is provided an injectable, sterile, ready to use, pyrogen-free pamidronate solution comprising a physiologically acceptable water soluble alkaline salt of pamidronate and a physiologically acceptable aqueous solvent having a concentration of between 0.1 and 100 mg/mL which has not been reconstituted from a lyophilized wherein the solution is provided in a sealed non-reactive container.

According to an aspect of the present invention, there is provided a process for producing a sterile, injectable, pyrogen-free, ready-to-use pamidronate solution comprising adding pamidronic acid to an aqueous solvent wherein the aqueous solvent contains sodium hydroxide, potassium hydroxide, or water soluble organic amines and placing the resulting solution in a non-reactive container.

Preferably, sodium hydroxide is mixed with pamidronic acid in a 2 to 1 molar ratio in an aqueous solvent to make pamidronate disodium. In a preferred embodiment, the solution is stored in plastic vials, with non-reactive stoppers such as Teflon™-coated or Teflon™-faced stoppers.

The invention provides for use of the solution to treat diseases selected from the group of tumour-induced hypercalcemia, Paget's disease, osteoporosis, bone metastases, and breast cancer.

### DETAILED DESCRIPTION OF THE INVENTION

Any physiologically acceptable alkaline salt of pamidronate that is water-soluble may be used for preparing the solution of the invention. Preferred salts are sodium and potassium. The disodium salt is the most preferred salt.

In a preferred embodiment, aqueous sodium hydroxide is added to a non-reactive mixing tank such as a polypropylene tank. Pamidronic acid is mixed with sodium hydroxide, in a 1:2 molar ratio, in an aqueous environment. Preferably, the required amount of sodium hydroxide is present in the sodium hydroxide solution prior to the addition of pamidronic acid. Any aqueous solvent that is physiologically acceptable in which pamidronate remains soluble may be used. The preferred aqueous solvent is water.

In this embodiment of the present invention, the aqueous sodium hydroxide solution is prepared by initially adding water to the polypropylene mixing tank. The required amount of sodium hydroxide is then added to the water and mixed until completely dissolved. The pamidronic acid in solid form is then added to the aqueous sodium hydroxide solution and mixed thoroughly until the pamidronic acid is completely dissolved. Preferably, the aqueous sodium hydroxide solution is continuously agitated while the pamidronic acid is added to the tank. Alternatively, the pamidronic acid is added to the tank without agitation and with agitation starting only after all pamidronic acid has been added.

The solution of the invention may also contain one or more additional components such as a preservative, a co-solubilizing agent, or any other desired agent. Suitable solvents include those that have acceptable particulate counts, such as water, or physiological saline. Tonicity adjustment agents in an amount that does not cause precipitation may be added, such as sodium chloride, dextrose, lactose, mannitol and the like.

Optionally, preservatives suitable for a physiological administration such as hydroxybenzoic acid esters, chlorobutanol and benzyl alcohol may be added. Although pH adjustment is not necessary for stability purposes, optionally, the pH may be adjusted within the range of from 6 to 10 using any known method of pH adjustment. Preferably, when pH adjustment is necessary, 10% phosphoric acid is used. The preferred pH of the pamidronate solution of the present invention is about 6.3 to about 6.7, more preferably about 6.4 to about 6.6, and most preferably about 6.5.

The concentration of the solution may be anywhere from 0.1 mg/mL to 100 mg/mL, preferably from about 1 to about 25 mg/mL and most preferably between about 3 to about 9 mg/mL. In the embodiments described in the Examples below, the pamidronate solutions of the present invention have concentrations of 3 mg/mL and 9 mg/mL.

The resulting solution may be filtered to remove particulate matter, and then is filled into a non-reactive container. “Non-reactive”, when used herein means that the material from which the container is constructed must not contain multivalent metal cations that can react with the pamidronate entity. The non-reactive containers used in the present invention are preferably made of plastic such as, for example, polypropylene, polyolefin, cycloolefin, polycarbonate, ABS resin, polyethylene, or PVC. Examples of suitable cycloolefin containers for use in the present invention are TopPac® containers (cycloolefin copolymers, amorphous thermoplastic) manufactured by Schott Corporation.

The non-reactive containers used in the present invention have any thickness that allows the containers to hold the pamidronate solution. Preferably, the non-reactive containers have a thickness of about 10 mm to about 50 mm, and most preferably about 10 mm to about 20 mm.

Alternatively, the non-reactive containers used in the present invention are constructed of glass that has been surface treated. The surface treatment of the inner surface of the glass container renders the glass container unreactive with the pamidronate solution. The surface treatment of the glass significantly reduces the ability of metal cations present in the glass from migrating into the pamidronate solution thereby causing degradation. Any suitable surface treatment that presents the metal cations in the glass from causing degradation of the pamidronate solution may be used in the present invention. Suitable surface treatments for glass containers useful in the present invention include, for example, ammonium sulfate, sulfur dioxide and ammonium chloride.

Preferably the non-reactive containers are vials. Also within the scope of the present invention is the use of non-reactive intravenous bags, and non-reactive ampoules, such as zirconium ampoules or form seal ampoules.

The non-reactive containers may be sealed using non-reactive stoppers to reduce contact between the pamidronate solution and potentially reactive surfaces that could lead to degradation. Preferred non-reactive stoppers are either Teflon™ coated or Teflon™ faced. Silicone rubber stoppers or other suitable non-reactive stoppers are contemplated. A non-reactive stopper useful in the present invention is a chlorobutyl rubber stopper that is Teflon™ coated manufactured by West Pharmaceutical Services.

Sterility of the product may be assured through making the product in aseptic conditions, or other methods for sterilization may be used. An advantage of the present invention is the ability to use terminal sterilization processes such as autoclaving. “Terminal sterilization”, when used herein, means steam sterilization by autoclaving using a process validated to deliver a minimum end of exposure Fo of 8 minutes and a maximum Fo of 15 minutes. The solution may be autoclaved according to methods known in the art. Alternatively, the solution may be passed through a sterilizing filter, such as a 0.22 micron Supor DCF capsule.

The solutions of the invention are characterized by good stability. Solutions have been found to be stable for long periods at room temperature. This is illustrated in the examples which follow.

The pharmaceutical compositions of the present invention are useful for treating any bone resorption disorders or conditions. Examples of these indications are tumor-induced hypercalcemia, conditions associated with increased osteoclast activity, predominantly lytic bone metastases and multiple myeloma as well as symptomatic Paget's disease of bone.

The composition of the present invention is designed to be diluted and administered as a slow intravenous infusion. The injectable solutions of the invention are administered according to a variety of possible dose schedules. Suitable dose schedules are for example 90 mg as a 2 hour infusion in 250 ml infusion solution or a maximum of 90 mg in 500 ml over 4 hours for patients with multiple myeloma or tumor induced hypercalcemia. The total dose for a treatment course may be given as a single infusion, or in multiple infusions spread over 2-4 consecutive days. The maximum dose should be 90 mg. The recommended total dose of pamidronate disodium injection for a treatment course for Paget's disease of the bone is 180-210 mg either administered as 6 doses of 30 mg once a week or 3 doses of 60 mg every second week following initiation with a 30 mg dose.

In light of the present disclosure, those skilled in the art will readily appreciate other methods and applications of the methods of the present invention.

The examples below are non-limiting and are merely representative of various aspects and features of the present invention.

With reference to the examples, the stability testing on the injectable solution was carried out by means of high performance liquid chromatography (HPLC) at the following experimental conditions:

### HPLC Assay Method

| | |
|---|---|
| Column(s)/temperature (if | Waters IC Pak Anion HR, |
| other than ambient): | 4.6 × 75 mm HPLC column or |
| | equivalent/35° C. |
| Mobile phase (specify | 0.0165% formic acid, pH = 3.5 |
| gradient program (if | |
| applicable): | |
| Detector/wavelength (if | Refractive Index |
| applicable): | |
| Flow rate: | 1.0 mL/min. |
| Injection volume: | 10 μL |

### EXAMPLE 1

Pamidronate disodium solution 3 mg/mL

| Composition | For 1 Vial (10 mL) | per mL |
|---|---|---|
| Pamidronic acid | 25.28 mg | 2.528 mg |
| Sodium | 8.61 mg | 0.861 mg |
| hydroxide NF | | |
| Mannitol USP | 470.0 mg | 47.0 mg |
| Water for | Q.S. to 10 mL volume | Q.S. to 1 mL volume |
| injection USP | | |
| Phosphoric acid NF | 10% for pH adjustment | 10% for pH adjustment |

### EXAMPLE 2

Pamidronate disodium solution 9 mg/mL

| Composition | For 1 Vial (10 mL) | per mL |
|---|---|---|
| Pamidronic acid | 75.82 mg | 7.582 mg |
| Sodium | 25.81 mg | 2.581 mg |
| hydroxide NF | | |
| Mannitol USP | 375.0 mg | 37.5 mg |
| Water for | Q.S. to 10 mL volume | Q.S. to 1 mL volume |
| injection USP | | |
| Phosphoric acid NF | 10% for pH adjustment | 10% for pH adjustment |

Water for injection USP was collected in a clean, non-reacting polypropylene mixing tank at room temperature. Sodium hydroxide NF was added to the water and mixed thoroughly until completely dissolved. Pamidronic acid was then added and mixed until completely dissolved. Mannitol USP was then added and completely dissolved. The pH was then adjusted to between 6.4 and 6.6 with 10% phosphoric acid. Water for injection USP was added to the final required volume.

The solution was filtered through a sterilizing 0.22 micron Supor-DCF filter. Volumes of 10 ml of the solution were distributed into plastic vials. The vials were then closed with Teflon™-faced/coated rubber stoppers and sealed, and steam sterilized by autoclaving using a process validated to deliver a minimum end of exposure Fo of 8 minutes and a maximum Fo of 15 minutes.

The stability of the solutions in the vials was tested after accelerated testing at 40° C. (accelerated stability controls) and at room temperature for a minimum of 6 months. The stability data obtained for the 3 mg/mL and 9 mg/mL concentrations, using HPLC for the determination of potency, are reported in the following Tables 1 and 2.

TABLE 1
INITIAL VALUES
Concentration: 3 mg/mL
Relative % Assay: 100.0%
pH: 6.62

| TEMPERATURE | |
|---|---|
| 25° ± 2° C./ | 40° ± 2° C./ |
| 60% ± 5% RH | 75% ± 5% RH |

| Time | Conc. | Rel. % | Conc. | Rel. % |
|---|---|---|---|---|
| (months) | mg/mL | Assay | mg/mL | Assay |
| 0 | 3.00 | 100.0 | 3.00 | 100.0 |
| 1 | — | — | 3.02 | 100.7 |
| 2 | — | — | 3.00 | 100.0 |
| 3 | 3.01 | 100.3 | 3.01 | 100.3 |
| 6 | 2.99 | 99.7 | — | — |

TABLE 2
INITIAL VALUES
Concentration: 9 mg/mL
Relative % Assay: 100.0%
pH: 6.49

| TEMPERATURE | |
|---|---|
| 25° ± 2° C./ | 40° ± 2° C./ |
| 60% ± 5% RH | 75% ± 5% RH |

| Time | Conc. | Rel. % | Conc. | Rel. % |
|---|---|---|---|---|
| (months) | mg/mL | Assay | mg/mL | Assay |
| 0 | 8.66 | 96.2 | 8.66 | 96.2 |
| 1 | — | — | 8.71 | 96.8 |
| 2 | — | — | 8.67 | 96.3 |
| 3 | 8.70 | 96.7 | 8.71 | 96.8 |
| 6 | 8.71 | 96.8 | — | — |

### EXAMPLE 3

This example illustrates the stability of the pamidronate solutions in non-reactive plastic containers in accordance with the present invention.

A pamidronate disodium 9 mg/mL formulation was prepared and filled into 10 mL polypropylene vials and cycloolefin vials (TopPac® cycloolefin copolymers, amorphous thermoplastic, manufactured by Schott Corporation). The vials were stoppered with West Teflon-coated stoppers. A portion of the polypropylene vials and all of the cycloolefin vials were then terminally sterilized at 121° C. for 18 minutes. A portion of the vials were then placed under accelerated storage conditions (40° C./10% RH) and a portion of the vials placed under room temperature storage conditions (25° C./30% RH).

The analysis of the physical properties of the pamidronate solutions at various time periods is set forth in the following table.

TABLE 3

| | | | 40° C./10% RH | | | | | 25° C./10% RH | | | |
|---|---|---|---|---|---|---|---|---|---|---|---|
| Vials/ | Test | Zero | 1M | 2M | | 3M | | 3M | | 10M | |
| TS or Non-TS | Parameter | Time | ↓ | ↑ | ↓ | ↑ | ↓ | ↑ | ↓ | ↑ | ↓ |
| Polypropylene | Visual Color | NT | CO | CO | CO | CO | CO | CO | CO | NT | NT |
| TS | Visual Clarity | NT | Clear | Clear | Clear | Clear | Clear | Clear | Clear | NT | Clear |
| | pH | 6.6 | 6.7 | NT | NT | NT | NT | NT | NT | NT | NT |
| | % Claim | NT | 107.4 | 107.2 | 100.8 | 105.3 | 104.9 | 100.2 | 106.1 | NT | 102.8 |
| | % phosphite | NT | ND | ND | ND | ND | ND | ND | ND | NT | NT |
| | % β alanine | <0.05 | <0.05 | <0.05 | <0.05 | NT | NT | NT | NT | <0.05 | NT |
| | % Total | <0.05 | <0.05 | <0.05 | <0.05 | NT | NT | NT | NT | <0.05 | NT |
| | Impurities | | | | | | | | | | |
| Polypropylene | Visual Color | NT | CO | CO | CO | CO | CO | CO | CO | NT | NT |
| Non-TS | Visual Clarity | NT | Clear | Clear | Clear | Clear | Clear | Clear | Clear | NT | Clear |
| | pH | 6.6 | 6.6 | NT | NT | NT | NT | NT | NT | NT | NT |
| | % Claim | 94.2 | 100.3 | 100.9 | 101.2 | 100.3 | 101.2 | 100.0 | 99.8 | NT | 105.1 |
| | % phosphite | NT | ND | ND | ND | ND | ND | ND | ND | NT | NT |
| | % β alanine | <0.05 | <0.05 | <0.05 | <0.05 | NT | NT | NT | NT | <0.05 | NT |
| | % Total | <0.05 | <0.05 | <0.05 | <0.05 | NT | NT | NT | NT | <0.05 | NT |
| | Impurities | | | | | | | | | | |
| TopPac | Visual Color | NT | CO | CO | CO | CO | CO | CO | CO | NT | NT |
| TS | Visual Clarity | NT | Clear | Clear | Clear | Clear | Clear | Clear | Clear | Clear | Clear |
| | pH | 6.6 | 6.5 | NT | NT | NT | NT | NT | NT | NT | NT |
| | % Claim | 101.8 | 100.2 | 100.6 | 100.9 | 99.8 | 99.9 | 99.8 | 99.6 | 102.9 | 103.0 |
| | % phosphite | NT | ND | ND | ND | NT | ND | ND | ND | NT | NT |
| | % β alanine | <0.05 | <0.05 | <0.05 | <0.05 | NT | NT | NT | NT | <0.05 | <0.05 |
| | % Total | <0.05 | <0.05 | <0.05 | <0.05 | NT | NT | NT | NT | <0.05 | <0.05 |
| | Impurities | | | | | | | | | | |

↑ = stored upright
↓ = stored inverted
CO = Colorless
NT = Not Tested
ND = Not Detected
TS = Terminally Sterilized

The pamidronate solutions of the present invention exhibited good stability for long-term shelf line and storage. The pamidronate solutions retained their potency and clarity. Moreover, the pamidronate solutions did not exhibit degradation as evidenced by the negligible level of impurities present.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An injectable, sterile, ready to use, pyrogen-free pamidronate solution comprising a physiologically accepted alkaline salt of pamidronate which is water soluble and a physiologically acceptable aqueous solvent having a concentration of between 0.1 and 100 mg/mL, wherein the solution is provided in a sealed non-reactive plastic container.

2. The solution according to claim 1, wherein the physiologically acceptable alkaline salt is selected from the group of sodium and potassium salts.

3. The solution according to claim 1, wherein the physiologically acceptable alkaline salt is the disodium salt.

4. The solution according to claim 3, wherein the concentration is between about 3 mg/mL and about 9 mg/mL.

5. The solution according to claim 4, wherein the concentration is 3 mg/mL.

6. The solution according to claim 4, wherein the concentration is 9 mg/mL.

7. The solution according to claim 4, wherein the sealed container is made of a material selected from the group of polypropylene, polyolefin, cycloolefin, polycarbonate, ABS resin, polyethylene, and PVC.

8. The solution according to claim 7, wherein the sealed container is polypropylene.

9. The solution according to claim 7, wherein the sealed container is closed with a non-reactive stopper.

10. The solution according to claim 9, wherein the stopper is selected from the group consisting of a Teflon-coated stopper and a Teflon-faced stopper.

11. The solution according to claim 7, further comprising a tonicity adjustment agent.

12. The solution according to claim 11, wherein the tonicity adjustment agent is selected from the group consisting of dextrose, lactose and mannitol.

13. The solution according to claim 8, wherein the solution has a pH of about 6.3 to about 6.7.

14. The solution according to claim 13, wherein the aqueous solvent is selected from the group consisting of water and physiological saline.

15. A process for producing a sterile, injectable, pyrogen-free, ready-to-use pamidronate solution comprising adding pamidronic acid to an aqueous solvent to form a pamidronate solution, wherein the aqueous solvent contains a compound selected from the group consisting of sodium hydroxide, potassium hydroxide or a water soluble organic amine, filling the pamidronate solution into non-reactive plastic containers, and sealing the containers.

16. The process according to claim 15, wherein the aqueous solvent contains sodium hydroxide.

17. The process according to claim 16, wherein sodium hydroxide is added to the aqueous solvent prior to the addition of pamidronic acid.

18. The process according to claim 17, wherein the molar ratio of sodium hydroxide to pamidronic acid is 2:1.

19. The process according to claim 18, wherein the sealed plastic container is a vial.

20. The process according to claim 19, wherein the sealed container additionally comprises a non-reactive stopper.

21. The process according to claim 18, further comprising adjusting the pH of the pamidronate solution to 6 to 10.

22. The process according to claim 21, wherein the pH of the pamidronate solution is adjusted to about 6.3 to about 6.7.

23. The process according to claim 22, further comprising passing the pamidronate solution through a sterilizing filter before filling the solution into non-reactive plastic containers.

24. The process according to claim 22, further comprising terminally sterilizing the sealed containers.

25. A process for manufacturing an aqueous solution of pamidronate, comprising adding pamidronic acid to an aqueous sodium hydroxide solution to form the disodium salt of pamidronate.

26. The process according to claim 25, wherein the molar ratio of sodium hydroxide to pamidronic acid is 2:1.

27. The process according to claim 26, wherein the required amount of sodium hydroxide is present in the aqueous sodium hydroxide solution prior to the addition of pamidronic acid.
